(19) United States
(12) Patent Application Publication
(10) Pub. No.: US 2015/0093331 A1
(43) Pub. Date: Apr. 2, 2015

(54) BIOMARKERS FOR BREAST CANCER

(71) Applicant: OncoLock Co., Ltd., Taipei City (TW)

(72) Inventor: Yeou-Guang Tsay, Taipei City (TW)

(73) Assignee: OncoLock Co., Ltd., Taipei City (TW)

(21) Appl. No.: 14/476,170

(22) Filed: Sep. 3, 2014

Related U.S. Application Data
(60) Provisional application No. 61/885,103, filed on Oct. 1, 2013.

(51) Int. Cl. G01N 33/574; C07K 16/18; C07K 14/47

(52) U.S. Cl. CPC G01N 33/57415; C07K 14/472; C07K 16/18; G01N 2333/4716; C07K 2317/24
USPC 424/9.1; 435/7.92; 530/395; 530/387.9; 530/387.3; 424/139.1; 436/501

(57) ABSTRACT

The present invention relates to biomarkers, methods and assay kits for the identification, monitoring and treatment of breast cancer.

### RELATED APPLICATION

The present application claims priority under 35 U.S.C. §119(e) to U.S. Provisional Patent Application No. 61/885,103, filed Oct. 1, 2013, the content of which is herein incorporated by reference in its entirety.

### TECHNOLOGY FIELD

The present invention relates to biomarkers, methods and assay kits for the identification, monitoring and treatment of breast cancer.

### BACKGROUND OF THE INVENTION

Breast cancer is the most common cancer in women and becomes a major cause of death from cancer among women worldwide. Needless to say, early detection of breast cancer provides the best chance for a cure. Some biomarkers, such as BRCA1, BRCA2 and Her-2/neu, have been identified as risk genes of breast cancer, which can be used to predict the risk for developing breast cancer in the lifetime. Further, mammography is used for detecting small breast tumors. However, there are currently no screening methods for diagnosing early-stage breast cancers, especially by blood test.

### BRIEF SUMMARY OF THE INVENTION

It is unexpectedly found that in breast cancer patients, the plasma protein complement factor H(CFH) changes in structure by proteolytic removal of the amino acid residue arginine at position 341 (Arg-341), leading to the production of two peptide fragments, the smaller one being about 40 kDa and the larger one being about 140 kDa, and such cleaved form of CFH is detectable specifically in breast cancer patients, even in early stage, but not in individuals free of breast cancer, including breast cancer survivors who become cancer-free after treatment. Accordingly, the cleaved form of the CFH protein resulting from proteolytic removal of Arg-341, can serve as a specific molecular marker for diagnosing breast cancer, even in early stage, and also for monitoring progression of breast cancer or therapeutic efficacy of a breast cancer treatment in a patient with breast cancer.

In one aspect, the present disclosure provides an assay method, comprising: (a) obtaining a biological sample from a subject; and (b) detecting a cleaved form of a complement factor H(CFH) protein in the sample by, e.g., a mass spectrometric assay or an immunoassay.

In some embodiments, the cleaved form of CFH is detected with an agent that specifically binds the cleaved form of CFH, e.g., an antibody. Such an antibody can specifically binds to (i) a peptide of SEQ ID NO:2, or (ii) a peptide of SEQ ID NO:3. For example, the antibody can bind to an epitope comprising the C-terminal Met residue of SEQ ID NO:2 or an epitope comprising the N-terminal Arg residue of SEQ ID NO:3, but the antibody does not bind to the intact form of CFH.

The biological sample to be examined in the methods described herein can be body fluid sample, including, but not limited to a sample from blood, urine, saliva, tear, cerebrospinal fluids, ascites, lymph or aspiration fluids e.g. nipple aspiration fluids. Typically, a blood sample can be whole blood or a faction thereof e.g. serum or plasma, heparinized or EDTA treated to avoid blood clotting. Alternatively, the biological sample can be a tissue sample or a biopsy sample.

In some embodiments, the assay method described herein can further comprise identifying the subject as having or at risk for breast cancer, if the cleaved form of CFH in the sample is elevated as compared to a reference value. In some examples, presence of the cleaved form of CFH in the sample as compared to the absence of the cleaved form of CFH in a control sample (e.g., the reference value being 0) is indicative of breast cancer occurrence or risk for developing the cancer. In other examples, an increased level of the cleaved form of CFH in the sample as compared to the reference value (e.g., the level of the cleaved CFH in a control sample) is indicative of breast cancer occurrence or risk.

Any of the methods described herein can further comprising optionally, treating the subject with an anti-breast cancer therapy, if the subject is identified as having breast cancer, wherein the anti-breast cancer therapy is surgery, radiotherapy, or chemotherapy. In some examples, the subject identified as having breast cancer can be subjected to chemotherapy, which may involve an anti-breast cancer drug selected from the group consisting of Abraxane, Anastrozole, Arimidex, Aromasin, Avastin, Docefrez, Docelaxel, Ellence, Epirubicin, Eribulin, Exemestane, Fareston, Faslodex, Femara, Fulvestrant, Gemcitabine, Gemzar, Halaven, Herceptin, Lxabepiline, Lxempra, Lapatinib, Letrozole, Megestrol, Paclitaxel, Tamoxifen, Taxotere, Toremifene, Trastuzumab, and Tykerb.

Alternatively or in addition, the method as described herein can further comprise applying a breast cancer diagnostic assay to a subject identified by the method described herein to confirm breast cancer occurrence.

In another aspect, the present disclosure provides a method for monitoring progression of breast cancer in a breast cancer patient, the method comprising: (a) obtaining a first biological sample from the patient at a first time point, (b) obtaining a second biological sample from the patient at a second time point, which is later than the first time point, (c) measuring the levels of a cleaved form of a complement factor H(CFH) protein in the first and second biological samples by a mass spectrometric assay or an immunoassay; and (d) determining breast cancer progression in the patient based on the levels of the cleaved form of CFH in the first and second biological samples, wherein an elevated level of the cleaved form of CFH in the second biological sample as compared to that in the first biological sample is indicative of breast cancer progression. In some examples, the method can further comprise assessing efficacy of the anti-breast cancer therapy on the patient, wherein a decrease of the level of the cleaved form of CFH after the treatment or over the course of the treatment indicates that the therapy is effective on the patient.

In some embodiments, the patient is subjected to an anti-breast cancer therapy and the first and second biological samples are obtained either before and after the treatment or during the course of the treatment.

In some embodiments, the cleaved form of CFH is detected with an agent that specifically binds the cleaved form of CFH, which can be an antibody, such as an antibody specifically binds to (i) a peptide of SEQ ID NO:2, or (ii) a peptide of SEQ ID NO:3. In one example, the antibody binds to an epitope comprising the C-terminal Met residue of SEQ ID NO:2 or an epitope comprising the N-terminal Arg residue of SEQ ID NO:3. Alternatively or in addition, the antibody does not bind to the intact form of CFH.

In yet another aspect, the present disclosure features an isolated antibody specifically binding to a cleaved form of a complement factor H(CFH) protein. In some embodiments, the antibody specifically binds to SEQ ID NO: 2 or 3. For example, the antibody can specifically bind to the C-terminal portion of SEQ ID NO: 2 or the N-terminal portion of SEQ ID NO: 3. In one example, the antibody binds to an epitope comprising the C-terminal Met residue of SEQ ID NO:2 or an epitope comprising the N-terminal Arg residue of SEQ ID NO:3. In another example, the antibody does not bind to the intact form of CFH.

In some examples, the antibody described herein can be a monoclonal antibody or a polyclonal antibody. In other examples, the antibody is a chimeric antibody or a humanized antibody.

Also within the scope of the present disclosure is a polypeptide or a complex comprising such, wherein the polypeptide comprises an amino acid sequence at least 90% (e.g., 95%, 97%, 99%, or above) identical to that of a cleaved form of CFH. In some embodiments, the cleaved form of CFH is SEQ ID NO:2, SEQ ID NO:3, or a complex form thereby. In one example, the polypeptide or a complex thereof comprises SEQ ID NO:2, SEQ ID NO:3. Such polypeptides or complexes thereof can be prepared by, e.g., recombinant technology.

In still another aspect, the present disclosure provides methods of using any of the antibodies described herein for treating or in vivo diagnosing breast cancer. When used for treating breast cancer, the antibody is conjugated to an anti-breast cancer agent (e.g., those described herein). When used for in vivo diagnostic purposes, the antibody can be conjugated to a detectable label suitable for diagnostic uses, which are known in the art. To perform such a method, an effective amount of a composition comprising the conjugated antibody can be administered to a subject in need of the treatment.

Also within the scope of the present disclosure is a cleaved form of CFH as described herein, which can be prepared by recombinant technology or chemical synthesis.

The details of one or more embodiments of the invention are set forth in the description below. Other features or advantages of the present invention will be apparent from the following detailed description of several embodiments, and also from the appended claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 showing the model that predicts that the protein products in plasma proteome secondary to proteolytic processing are cancer-specific biomarkers. Polypeptide chains (thick black lines) in plasma are rich of disulfide bonds (thin lines), which are classified as intrachain and interchain ones based on the number of the thiol-containing polypeptide chains involved in the linkage. Particularly, interchain disulfides, connecting two different polypeptide chains, can help stabilize proteins that are processed by cancer-specific proteases (grey triangle) and keep plasma proteins containing proteolytic footprints stay in the plasma. By quantitatively analyzing these protein products, we can detect the presence of cancer-specific protease activity, which can serve as a method for specific diagnosis of human cancers.

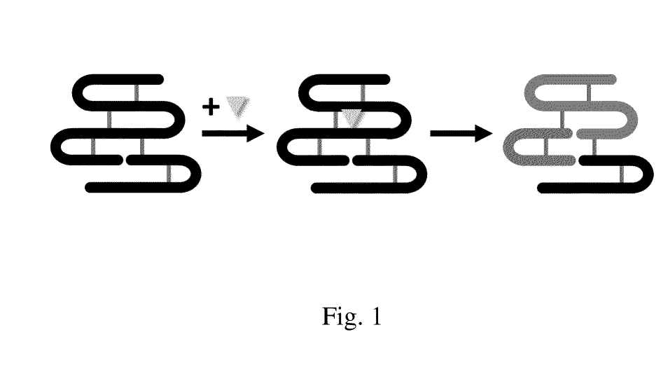

FIG. 2 showing western blot analyses of plasma samples from a breast cancer patient (P) and a normal individual (C) with antibodies against complement factor H. Proteins in the plasma samples from tested subjects were electrophoresed under non-reducing (left panel) or reducing condition (right panel). The numbers to the left, expressed in kilodaltons (kDa), are the positions of molecular mass markers. The full circle denotes the intact complement factor H polypeptide, which presumably becomes large (major circular sector) and small (minor circular sector) fragments by proteolytic processing. These two fragments remains connected by disulfide bond linkages (thin black line) in non-reducing condition while becoming disconnected under reducing conditions.

FIG. 3 showing the tandem mass spectrum of doubly charged ³³²HGGLYHENM³⁴⁰ (SEQ ID NO:7) from the tryptic Lys-C digest of 40 kDa CFH fragment. The numbers flanking the peptide sequence indicate the end positions of this peptide in intact factor H. Identified b and y ions are marked by the numbers above and below the peptide sequence, respectively. Sequest analyses showed a correlation score of 1.40 when this tandem mass spectrum was matched with this sequence.

FIG. 4 showing the tandem mass spectrum of triply charged ³²⁰CTLPKCDYPDIKHGGLYHENM³⁴⁰ (SEQ ID NO:5) from the Arg-C digest of 40 kDa CFH fragment. The numbers flanking the peptide sequence indicate the end positions of this peptide in intact factor H. Identified b and y ions are marked by the numbers above and below the peptide sequence, respectively. VP denotes the loss of a 105.0578-Da vinylpyridine group, with or without concurrent proton release. Sequest analyses showed a correlation score of 3.53 when this tandem mass spectrum was matched with this sequence.

FIG. 5 showing the tandem mass spectrum of doubly charged ³⁴²RPYFPVAVGK³⁵¹ (SEQ ID NO:6) from the Lys-C digest of 140 kDa CFH fragment. The numbers flanking the peptide sequence indicate the end positions of this peptide in intact factor H. Identified b and y ions are marked by the numbers above and below the peptide sequence, respectively. Sequest analyses showed a correlation score of 3.06 when this tandem mass spectrum was matched with this sequence.

FIG. 6 showing schematics summarizing the mass spectrometric results evidencing that fragments of complement factor H (SEQ ID NO:7) seen in breast cancer patients result from proteolytic removal of Arg-341. The domain structure of complement factor H, is shown at the top of the amino acid sequence of its CCP-6, one of the twenty complement control protein (CCP) modules in this protein. The numbers below the domain structure indicate the start and end positions of CCP-6 module. Those numbers to the right of the CCP-6 sequence specify the residue positions in intact factor H. The Cys residues involved in the first (solid-lined arrows) and second disulfide bonds (pointed-lined arrows) are indicated. The removal of Arg-341 is deduced by observation of Lys-C (dashed line), Arg-C (pointed line) and tryptic peptides (solid lines) in tandem mass spectrometric analyses.

FIG. 7 showing western blot analyses of plasma samples from a breast cancer patient (P) and a normal individual (C) using anti-BCPM 1 (left panel) or anti-BCPM2. The C-terminus of the 40 kDa proteolytic fragment is designated as breast cancer proteolytic marker 1 (BCPM1) and the N-terminus of the 140 kDa fragment is named breast cancer proteolytic marker 2 (BCPM2). Plasma proteins in the plasma samples from tested subjects were electrophoresed under reducing condition. The numbers to the left, expressed in kilodaltons (kDa), indicate where molecular mass markers migrated in the same gel.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

As used herein, the term “polypeptide” refers to a polymer composed of amino acid residues linked via peptide bonds. The term “protein” typically refers to relatively large polypeptides (e.g., containing more than 300, 400, 500, 600, 700, 800, 900, 1000, 1100, or 1200 amino acid residues). The term “peptide” typically refers to relatively short polypeptides (e.g., containing up to 150, 125, 100, 80, 60, 50, 30, or amino acid residues). Amino acids can be expressed by three letters or one letter as known in the art.

As used herein, a biological marker (or called biomarker or marker) is a characteristic that is objectively measured and evaluated as an indicator of normal or abnormal biologic processes, diseases, pathogenic processes, or responses to treatment or therapeutic interventions. Markers can include presence or absence of characteristics or patterns or collections of the characteristics which are indicative of particular biological processes. The biomarker measurement can increase or decrease to indicate a certain biological event or process. A marker is primarily used for diagnostic and prognostic purposes. However, it may be used for therapeutic, monitoring, drug screening and other purposes described herein, including evaluation the effectiveness of a cancer therapeutic.

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±10% around the cited value.

As used herein, the terms “subject,” “individual” and “patient,” used interchangeably herein, refer to any mammalian subject for whom diagnosis, prognosis, treatment, or therapy is desired, particularly humans. Other subjects may include cattle, dogs, cats, guinea pigs, rabbits, rats, mice, horses, and so on.

As used herein, the term “diagnosis” as used herein generally includes determination as to whether a subject is likely affected by a given disease, disorder or dysfunction. The skilled artisan often makes a diagnosis on the basis of one or more diagnostic indicators, i.e., a marker, the presence, absence, or amount of which is indicative of the presence or absence of the disease, disorder or dysfunction.

As used herein, an “aberrant level” means a level that is increased compared with the level in a subject free from cancer or a reference or control level. For examiner, an aberrant level can be higher than a reference or control level by more than 10%, 20%, 30%, 40%, 50%, 60%, 70%, 80%, 90% or 100%. A reference or control level can refer to the level measured in normal individuals or sample types such as tissues or cells that are not diseased.

As used herein, a biological sample to be analyzed by any of the methods described herein can be of any type of samples obtained from a subject to be diagnosed, including a sample from blood, urine, saliva, tear, cerebrospinal fluids, ascites, lymph or aspiration fluids e.g. nipple aspiration fluids. Typically, a blood sample can be whole blood or a faction thereof e.g. serum or plasma, heparinized or EDTA treated to avoid blood clotting. Alternatively, the biological sample can be a tissue sample or a biopsy sample.

The present disclosure is based at least in part on the identification of a novel reliable breast cancer biomarker, i.e., a cleaved form of CFH. As demonstrated in the examples below, this biomarker unexpectedly, allows for screening of early-stage breast cancer patients (e.g., stage 0 patients) using blood samples. Thus, described herein are a rapid and accurate working screening method for identifying subjects who have, suspected of have, or at risk for breast cancer. The diagnostic methods described herein can be applied as an initial, routine screening method to any subject (e.g., human female subjects) to identify those that are in early-stage breast cancer or those who are at risk for developing breast cancer.

I. Cleaved Form of CFH as a Novel Breast Cancer Biomarker

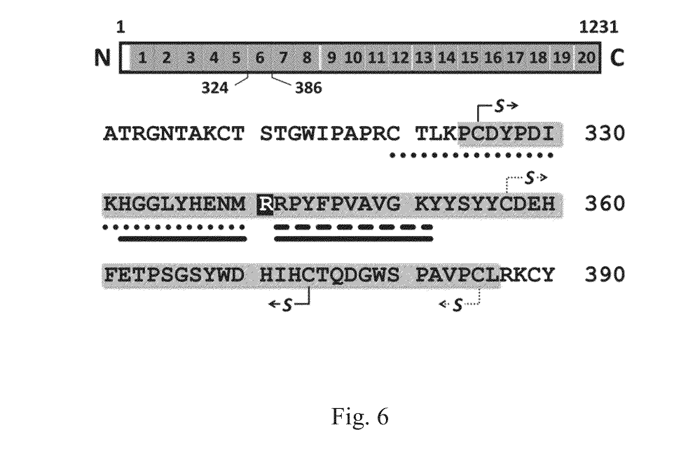

Complement factor H(CFH), also known as complement control protein (CCP), is a single-chain serum glycoprotein having the molecular weight of about 155 kDa, which regulates the formation and function of complement C3 and C5 convertase enzymes. Complement factor H is composed of 20 short consensus repeat (SCR) modules, each containing about 60 amino acids. The amino acid sequence within each single SCR is highly conformed conserved, including four cysteine residues which are required for disulfide-bond formation. FIG. 6 shows an exemplary domain structure of the CFH protein, including 20 SCR modules, CCP-1 to CCP-20. The amino acid sequence of the CFH protein and structure are well known in the art. For example, an exemplary human CFH protein is set forth as SEQ ID NO: 1. See examples below.

As noted herein, the present disclosure is based on the unexpected discovery that the presence and level of CFH in cleaved form correlates with breast cancer occurrence and development. As used herein, the term “cleaved form of CFH” or “cleaved CFH” refers to any CFH fragments or polypeptide complex thereof resulting from proteolysis of CFH. For example, a cleaved CFH can be produced via the removal of the amino acid residue at the position corresponding to Arg-341 in SEQ ID NO:1, leading to the production of two CFH fragments, the smaller one having SCR modules 1 to 5 with methionine at position 340 (Met-340) as the C-terminal residue (e.g., SEQ ID NO:2), and the larger one having SCR modules 6 to 20 with arginine at position 342 (Arg-342) as the N-terminal residue (e.g., SEQ ID NO:3). In certain embodiments, the smaller fragment has a molecular weight of about 40 kDa and the larger fragment has a molecular weight of about 140 kDa. In some examples, the cleaved form of CFH is the polypeptide of SEQ ID NO:2. In other examples, the cleaved form of CFH is the polypeptide of SEQ ID NO:3. In yet other examples, the cleaved form of CFH is the complex formed by SEQ ID NO:2 and SEQ ID NO:3.

Any of the cleaved form of CFH, or a complex form thereby, is also within the scope of the present disclosure. Such a cleaved form of CFH may be prepared by a conventional method, e.g., recombinant technology, using a suitable host cell (e.g., E. coli, yeast, mammalian, insect, or cell-free host system) or synthesis methods. In some examples, the cleaved CFH polypeptide differs from the naturally occurring counterpart in at least one post-translational modification such as glycosylation. The cleaved form of CFH can be mixed with a pharmaceutically acceptable carrier (e.g., a carrier that does not co-exist with the cleaved CFH in nature or a non-naturally occurring carrier) to form a composition for, e.g., pharmaceutical uses.

II. Assays for Detecting and Measuring the Levels of Cleaved CFH

The presence and level of a cleaved form of CFH in a biological sample can be determined by any routine technology. In some embodiments, the presence and/or level of a cleaved form of CFH can be determined by mass spectrometry, which allows direct measurements of the analytes with high sensitivity and reproducibility. A number of mass spectrometric methods are available. Examples of mass spectrometry include, but are not limited to, matrix-assisted laser desorption ionization/time of flight (MALDI-TOF), surface-enhanced laser desorption ionisation/time of flight (SELDI-TOF), liquid chromatography-mass spectrometry (LC-MS), liquid chromatography tandem mass spectrometry (LC-MS-MS), and electrospray ionization mass spectrometry (ESI-MS). One certain example of this approach is tandem mass spectrometry (MS/MS), which involves multiple steps of mass selection or analysis, usually separated by some form of fragmentation.

In other embodiments, the presence and/or level of a cleaved form of CFH can be determined by an immunoassay. Examples of the immunoassays include, but are not limited to, western blot, enzyme-linked immunosorbent assay (ELISA), radioimmunoassay (RIA), radioimmunoprecipitation assay (RIPA), immunofluorescence assay (IFA) and electrochemiluminescence (ECL).

In some examples, the presence and/or level of a cleaved form of CFH can be determined using an agent specifically recognizes the cleaved CFH, such as an antibody that specifically binds to the cleaved CFH.

(i) Antibodies Specifically Binding to Cleaved CFH

As used herein, the term “antibody” refers to an intact immunoglobulin or fragment thereof, and encompasses any polypeptide comprising an antigen-binding domain or an antigen-binding fragment that specifically binds to a particular antigen. The term includes but is not limited to monoclonal, monospecific, polyclonal, polyspecific, humanized, human, single-chain, chimeric, synthetic, recombinant, mutated, and hybrid antibodies. A suitable antibody can be prepared according to a method as known in the art. Such antibodies may not be naturally occurring (e.g., would not be produced in nature without human act).

An intact or complete antibody comprises two heavy chains and two light chains. Each heavy chain consists of a variable region (V_{H}) and a first, second and third constant regions (C_{H}1, C_{H}2 and C_{H}3), while each light chain consists of a variable region (V_{L}) and a constant region (C_{L}).

The term “antigen-binding domain” or “antigen-binding fragment” refers to a portion or region of an entire antibody molecule that is responsible for antigen binding. The portion of the antigen that is specifically bound or recognized by the antibody is called the “epitope.” An antigen-binding domain may comprise the heavy chain variable region (V_{H}) and the light chain variable region (V_{L}); however, it does not have to comprise both. The variable region in both chains typically contains three hypervariable regions called the complementarity determining regions (CDRs). The three CDRs are interrupted by framework regions (FRs), which are more highly conserved than the CDRs. The constant regions of the heavy and light chains are not responsible for antigen binding, but exhibit various effector functions. Antibodies are classified based on the amino acid sequence of the constant region of their heavy chain. The five major classes or isotypes of antibodies are IgG, IgM, IgA, IgD and IgE, which are characterized by the presence of the constant regions of the heavy chains, gamma, mu, alpha, delta and epison, respectively. Examples of antigen-binding fragments of an antibody include: (1) a Fab fragment, a monovalent fragment having the V_{L}, V_{H}, C_{L} and C_{H}1 domains; (2) a F(ab′)₂ fragment, a bivalent fragment having two Fab fragments linked by a disulfide bridge at the hinge region, i.e. a dimer of Fab; (3) a Fv fragment having the V_{L} and V_{H} domains of a single arm of an antibody; (4) an isolated complementarity determining region (CDR); (5) a single chain Fv (scFv), a single polypeptide chain composed of a V_{H} domain and a V_{L} domain through a peptide linker; and (6) a (scFv)₂, comprising three peptide chains: two V_{H} domains linked by a peptide linker and bound by disulfide bridges to two V_{L} domains.

The term “human antibody” includes antibodies having variable and constant regions corresponding substantially to, or derived from human germline immunoglobulin sequences. The human antibodies of the invention, however, may include amino acid residues not encoded by human germline immunoglobulin sequences (e.g., mutations introduced by random or site-specific mutagenesis in vitro or by somatic mutation in vivo), for example in the CDRs. Particularly, the human antibody may have at least one, two, three, four, five, or more positions replaced with an amino acid residue that is not encoded by the human germline immunoglobulin sequence.

In some embodiments, the antibody to be used in any of the methods described herein is an antibody that specifically binds to a cleaved form of CFH, e.g., the CFH fragment of SEQ ID NO:2, the CFH fragment of SEQ ID NO:3, or a complex formed by these two fragments.

An antibody that “specifically binds to a target (e.g., a cleaved form of CFH) or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a cleaved form of CFH or an epitope therein is an antibody that binds this target antigen with greater affinity, avidity, more readily, and/or with greater duration than it binds to other antigens or other epitopes in the same antigen, e.g., the full-length CFH. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

In some embodiments, the antibody for use in the methods described herein specifically binds to a fragment of CFH that consists of short consensus repeat (SCR) modules 1 to 5 with methionine at position 340 (Met-340) as the C-terminal residue. In other embodiments, the antibody specifically binds to a fragment of CFH that consists of SCR modules 6 to 20 with arginine at position 342 (Arg-342) as the N-terminal residue. In some embodiments, the antibody for use in the methods described herein specifically binds to SEQ ID NO: 2, or SEQ ID NO:3. In other examples, the antibody specifically binds to a complex formed by SEQ ID NO:2 and SEQ ID NO:3. Such an antibody may not bind to the full-length CFH. In one example, the antibody specifically binds to the C-terminal portion of SEQ ID NO: 2 or the N-terminal portion of SEQ ID NO: 3. For example, the antibody may bind to an epitope that comprises the C-terminal amino acid residue of SEQ ID NO:2 or the N-terminal amino acid residue of SEQ ID NO:3.

Any of the antibodies described herein is also within the scope of the present disclosure.

(ii) Antibody Preparation

Antibodies capable of binding cleaved CFH as described herein can be made by any method known in the art. See, for example, Harlow and Lane, (1988) Antibodies: A Laboratory Manual, Cold Spring Harbor Laboratory, New York.

In some embodiments, antibodies specific to a target antigen (e.g., a cleaved form of CFH such as SEQ ID NO:2 or SEQ ID NO:3) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody-producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of binding to a cleaved CFH. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy detecting a cleaved CFH. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)₂ fragments can be produced by pepsin digestion of an antibody molecule, and Fab fragments that can be generated by reducing the disulfide bridges of F(ab′)₂ fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single-chain antibodies (U.S. Pat. Nos. 4,946,778 and 4,704,692) can be adapted to produce a phage or yeast scFv library and scFv clones specific to a cleaved CFH can be identified from the library following routine procedures. Positive clones can be subjected to further screening to identify those that bind to a cleaved CFH.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of a cleaved CFH polypeptide have been replaced (swapped) with sequences from a closely related, but antigenically distinct protein (such as another member of the neurotrophin protein family). By assessing binding of the antibody to the mutant CFH, the importance of the particular antigen fragment to antibody binding can be assessed. Antibodies binding to desired epitopes can be identified via epitope mapping.

III. Diagnosis and Prognosis Applications of the Novel Breast Cancer Marker

As noted herein, cleaved form of CFH due to proteolysis was found, unexpectedly, to be associated with breast cancer occurrence and development. Thus, described herein are methods for diagnosing breast cancer patients at various disease stages, monitoring breast cancer progression, and assessing breast cancer treatment efficacies, using a cleaved form of CFH as a reliable biomarker. As noted above, the diagnostic method described herein can be applied as initial screening to any female subject to identify those having very early-stage breast cancer. A subject to be analyzed by the method can be any female mammal subject, such as human subjects. In some examples, the subject may exhibit one or more symptoms associated with breast cancer. In other examples, the subject may be at risk for developing breast cancer, e.g., carrying a breast cancer risk gene (such as mutations in BRCA1 and/or BRCA2 genes) or having family history of breast cancer, prostate cancer, melanoma, and pancreatic cancer. In yet other examples, the subject is asymptomatic. In yet other examples, the diagnostic method described herein can be used as a routine screening assay on a healthy female subject, e.g., a woman over 20, 35, 40, 45, 50 or 55, for monitoring potential breast cancer risk or development.

To perform the methods described herein, a biological sample can be obtained from a subject in need (e.g., a human patient who does not exhibit any symptoms of breast cancer, or a human patient having, suspected of having, or at risk for breast cancer) and a cleaved form of CFH or the level thereof in the biological sample can be detected or measured via any methods known in the art, such as those described herein, e.g., mass spectrometry and immunoassays. A biological sample can be a biofluid sample, such as a blood sample or a plasma sample. The detection of the cleaved CFH may be quantitative or qualitative. Any effective method in the art for measuring the presence/absence, level or activity of a marker is included in the invention. It is within the ability of one of ordinary skill in the art to determine which method would be most appropriate for measuring a specific marker.

In one embodiment, a sample obtained from a subject in need is tested for the presence or absence of a cleaved CFH. If a cleaved CFH is detected in a sample obtained from a subject in need, the subject is identified as having or at risk for breast cancer.

In some embodiments, the level of a cleaved CFH in a sample obtained from a candidate subject can be compared with a reference value to determine whether the candidate subject has or is at risk for breast cancer. The reference value represents the level of the cleaved CFH in a control sample. The control sample may be obtained from a subject (e.g., a female subject) who is free of breast cancer. Alternatively, the control sample may be obtained from a pool of such subjects. Optionally, these control subjects match with the candidate subject in, e.g., age, gender, and/or ethnic background. Preferably, the control sample and the biological sample of the candidate subject are of the same type. In some embodiments, the candidate subject may be identified as having, suspected of having, or at risk for breast cancer if a cleaved CFH is detected, or the level of a cleaved CFH is elevated as compared with the reference value (e.g., is higher than the reference value by about 10% or more). In some examples, the level of cleaved CFH in a control sample is undetectable in a control sample (the reference value being 0) using a routine assay, such as those described herein. In that case, detection of the cleaved CFH in a biological sample from a subject using the same assay indicates that the subject has, suspected of having, or at risk for breast cancer.

In some embodiments, the levels of a cleaved CFH in multiple samples from a candidate subject (e.g., a breast cancer patient) can be measured to determine disease progression. For example, at least two biological samples (e.g., serum samples or plasma samples) can be obtained from a candidate subject at different time points. The levels of a cleaved CFH in the at least two biological samples can be measured as described herein. If a trend of increase in the cleaved CFH level is observed over time (e.g., the level of a cleaved CFH in a later obtained sample is higher than that in an earlier obtained sample), the subject is identified as having, suspected of having, or at risk for breast cancer. If the subject is a breast cancer patient, an increased trend in the level of a cleaved CFH is indicative of breast cancer progression.

After a subject such as a human patient has been identified as having, suspected of having, or at risk for breast cancer, such a subject can be subjected to a further test (such as a conventional physical examination, including imaging tests, e.g., X-ray mammograms, magnetic resonance imaging (MRI) or ultrasound, nipple discharge exam or biopsy by needle or surgery) to confirm disease occurrence and/or determine the stage or types of breast cancer.

In some embodiments, the methods described herein can further comprise treating the breast cancer patient to at least alleviate a symptom associated with the disease. Such a treatment can be any conventional anti-breast cancer therapy, including ratiotherapy, chemotheray, and surgery. Exemplary anti-breast cancer chemotherapeutic agents include, but are not limited to, Abraxane, Anastrozole, Arimidex, Aromasin, Avastin, Docefrez, Docelaxel, Ellence, Epirubicin, Eribulin, Exemestane, Fareston, Faslodex, Femara, Fulvestrant, Gemcitabine, Gemzar, Halaven, Herceptin, Lxabepiline, Lxempra, Lapatinib, Letrozole, Megestrol, Paclitaxel, Tamoxifen, Taxotere, Toremifene, Trastuzumab, and Tykerb.

In yet other embodiments, provided here are methods for assessing the efficacy of a breast cancer treatment in a breast cancer patient. For example, multiple biological samples can be obtained from a breast cancer patient who is undergoing a breast cancer treatment over the course of the treatment. If a trend of decrease in the cleaved CFH level over the course of the treatment, i.e., the level of a cleaved CFH in a later obtained biological sample is lower than the level of the cleaved CFH in an earlier obtained biolocial sample, it indicates that the treatment is effective on the breast cancer patient. On the other hand, if the level of the cleaved CFH remains the same or increases over the course of the treatment, it indicates that the treatment might not be effective on that patient.

If a breast cancer therapy is determined to be ineffective on a breast cancer patient, treatment strategy can be adjusted, e.g., increasing drug dosage, treatment frequency, or change to a more suitable therapy.

IV. Kits

Also provided is a kit for performing the method of the invention, which comprises the antibody as described herein. The kit can further comprise instructions for using the kit to detect the presence or amount of the CFH protein with removal of Arg-341 or the resultant peptide fragment, for detecting breast cancer, and also for monitoring progression of breast cancer or therapeutic efficacy of a treatment in a patient with breast cancer.

Moreover, the present invention provides a method for treating a subject having breast cancer, comprising administering to said subject an amount of an antibody as described herein, linked with an anti-cancer agent.

Further provided is a method for performing an in vivo diagnosis in a subject in need, comprising administering to said subject an amount of an antibody as described herein, linked with an anti-cancer agent.

V. Use of Anti-Cleaved CFH Antibodies in Treating and Diagnosing Breast Cancer

Any of the antibodies described herein that specifically bind to a cleaved form of CFH can be used in treating breast cancer or in in vivo diagnosis of breast cancer. For example, the antibody can be conjugated to an anti-breast cancer agent (such as those described above) for use in treating breast cancer. Alternatively, the antibody can be conjugated to a detectable label (e.g., an in vivo imaging agent as known in the art) for diagnostic purposes.

To practice the diagnostic or treatment method disclosed herein, an effective amount of a pharmaceutical composition comprising an anti-cleaved CFH antibody, which is conjugated to either an anti-breast cancer agent or a detectable label, can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution.

The subject to be treated by the diagnostic or treatment methods described herein can be a female mammal, more preferably a human female subject. Mammals include, but are not limited to, farm animals, sport animals, pets, primates, horses, dogs, cats, mice and rats. A human subject who needs the treatment may be a human patient having a breast cancer, suspected of having a breast cancer, or at risk for breast cancer. Such a patient can be identified by routine medical procedures or the diagnostic method described herein.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration of breast cancer. Alternatively, sustained continuous release formulations of an anti-cleaved CFH may be appropriate. Various formulations and devices for achieving sustained release are known in the art.

The conjugated antibodies may be mixed with suitable pharmaceutically acceptable carriers to form suitable formulations depending upon the delivery route.

Injectable compositions may contain various carriers such as vegetable oils, dimethylactamide, dimethylormamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1

### Specific Proteolytic Fragments of Complement Factor H in Breast Cancer Patients

First, through a modified two-dimensional gel electrophoresis system (data not shown), we found that two fragments of complement factor H (factor H or CFH protein) in the plasma from certain breast cancer patients. Factor H contains twenty sushi domains, which are also named complement control protein (CCP) modules or short consensus repeats (SCR). Through LC-MS/MS analyses of their tryptic digests, we found that the smaller species, with a molecular mass of about 40 kDa, contained the amino acid sequence from CCP-1 to CCP-5 modules, while the larger 140 kDa fragment comprising the sequences of CCP-6 to CCP-20 (FIG. 6). These data suggested that these two CFH polypeptides could be the product of proteolytic processing.

In order to verify these results as well as to further characterize these protein fragments, we performed western blotting to analyze plasma proteins with antibodies against CFH protein.

Western Blot Analyses

Proteins in plasma samples collected from human subjects were mixed with 6× Laemmli sample buffer, containing 120 mM Tris-HCl, pH 6.8, 2% SDS (w/v) and 10% sucrose (w/v). For some samples, 1% of 2-mercaptoethanol was further included. Following electrophoretic separation, the proteins were transferred to Immobilon PVDF membranes (Millipore Corp.) using standard techniques. Blotted membranes were blocked with blocking buffer containing 1% of goat serum for 1 h at room temperature and then incubated with mouse anti-factor H (Abnova). After three washes with Tris-buffered saline containing 0.1% Tween 20, the membranes were incubated with peroxidase-conjugated goat anti-mouse IgG (Sigma-Aldrich). The chemiluminescence was detected using LAS-4000 (Fujifilm, Japan).

Results

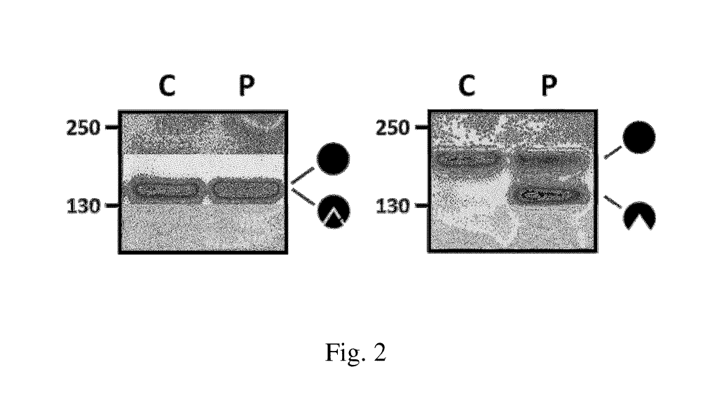

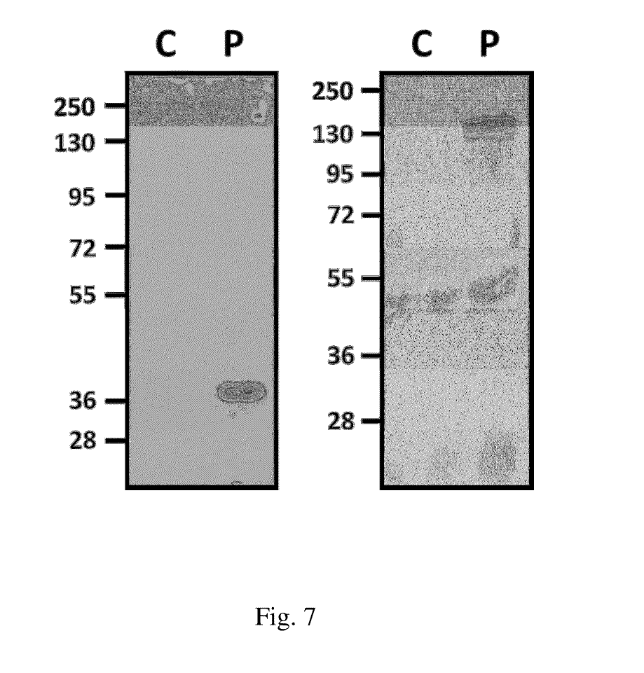

Western blot analyses showed that complement factor H in the plasma from normal individuals and breast cancer patients migrated like a polypeptide of about 150 kDa under non-reducing conditions. This gel mobility is consistent with the known length of this polypeptide. There was no apparent difference in factor H among these samples. However, when this protein was subjected to reducing SDS-PAGE analyses, for normal individuals, it migrated like a polypeptide of about 190 kDa. In the same electrophoretic analyses (reduced condition), one additional factor H species, about 140 kDa in size, was detected for breast cancer patients. We also employed two-dimensional difference gel electrophoresis (2D-DIGE), this detection is always concurrent with the appearance of 40 kDa CFH species (data not shown). We have used the same assay to examine this plasma protein for the same patient following operation and chemotherapy. Intriguingly, this 140 kDa species disappeared when the cancer became absent. These data together suggest that 40- and 140 kDa species are specifically present in the plasma from these cancer patients and are likely good indicators of the disease status (FIG. 2 and FIG. 7). Since these two species were not seen in the non-reducing analyses for control individuals, they are likely connected to each other through disulfide linkage and thus have the gel mobility of the intact polypeptide under non-reducing conditions. In short, these data support our finding that specific proteolytic fragments of complement factor H can be used as a diagnostic marker for breast cancer and an indicator to monitor the status of the diseases.

### Example 2

### Structural Analysis of Specific Proteolytic Fragments of Complement Factor H in Breast Cancer Patients

In order to characterize whether the 40- and 140 kDa species indeed resulted from proteolytic processing of complement factor H, we employed liquid chromatography-tandem mass spectrometry (LC-MS/MS) to examine the structural details of these two factor H fragments.

To map their ends of these two fragments, we further prepared their tryptic, Arg-C and Lys-C digests and subjected these digests to LC-MS/MS analyses.

In-Gel Protein Digestion

The gel piece containing the studied polypeptides was washed in 1 ml of 25 mM NH₄HCO₃ for 10 minutes and then dehydrated with 1 ml of 25 mM NH₄HCO₃/50% acetonitrile for 10 minutes. The wash solution was discarded and the gel pieces were dried in a vacuum evaporator centrifuge SpeedVac (Sovant) for 20 minutes. The dried gel pieces were incubated with 1% of β-mercaptoethanol for 20 minutes and 5% of 4-vinlpyridine was added for another 20-min incubation at dark. The gel piece was washed with 1 ml of 25 mM NH₄HCO₃ for 10 minutes, followed by treatment with 1 ml of 25 mM NH₄HCO₃/50% acetonitrile for 10 minutes. The gel piece was completely dried in SpeedVac and then incubated with 50 μl of 25 mM NH₄HCO₃ containing 50 ng of trypsin per sample. After overnight incubation, the solution was transferred to a new tube and the gel was extracted with 300 μl of 25 mM NH₄HCO₃ and 25 mM of NH₄HCO₃/50% acetonitrile sequentially. The protein digest and two extracts were pooled together and dried completely. These samples were stored at −20° C. until further use.

Mass Spectrometric Analyses

The protein digest was analyzed in LTQ-Orbitrap hybrid tandem mass spectrometer (ThermoFisher, USA) in-lined with Agilent 1200 nanaoflow HPLC system. The HPLC system was equipped with LC packing C18 PepMap100 (length: 5 mm; internal diameter: 300 μm; bead size: 5 μm) as the trap column and heat-pulled capillary in-house packed with 5 μm C18 beads (YMC ODS-AM) as the tip column. The mobile phase consisted of (A) 0.1% formic acid in water and (B) 0.1% formic acid in acetonitrile. Ten MS/MS scans with LTQ were collected following a full MS scan with Orbitrap. In-house program were used to extract the MS/MS information as well as to calculate the charges and masses of the precursor ions. Using TurboSequest program, all MS/MS data were searched against a human protein database, containing ˜440,000 protein entries, downloaded from National Center for Biotechnology Information (NCBI) on 5 May 2010. Tandem mass spectra were annotated using in-house programs written using Microsoft Excel 2013 VBA.

Results

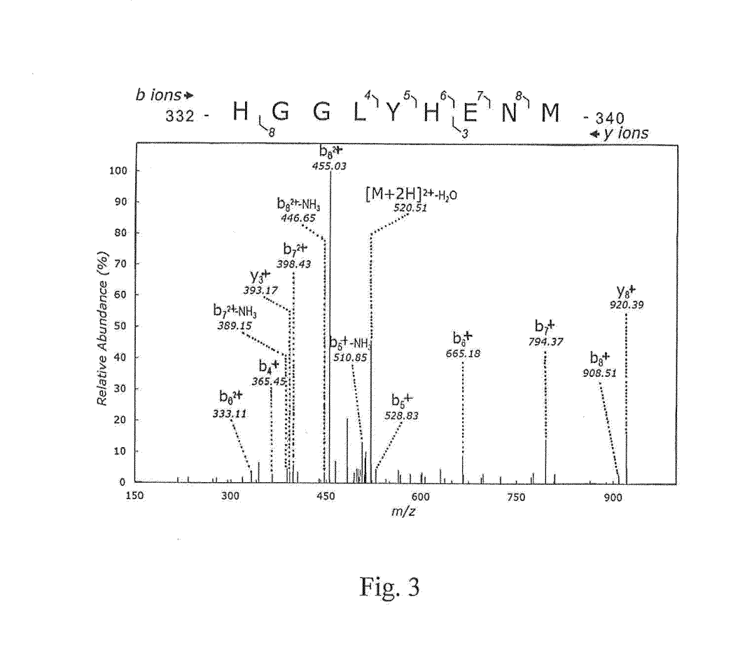

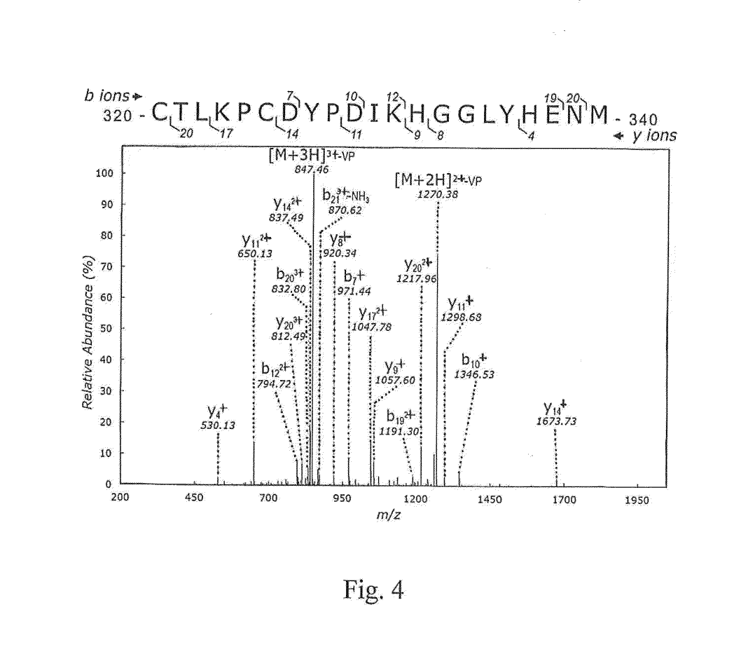

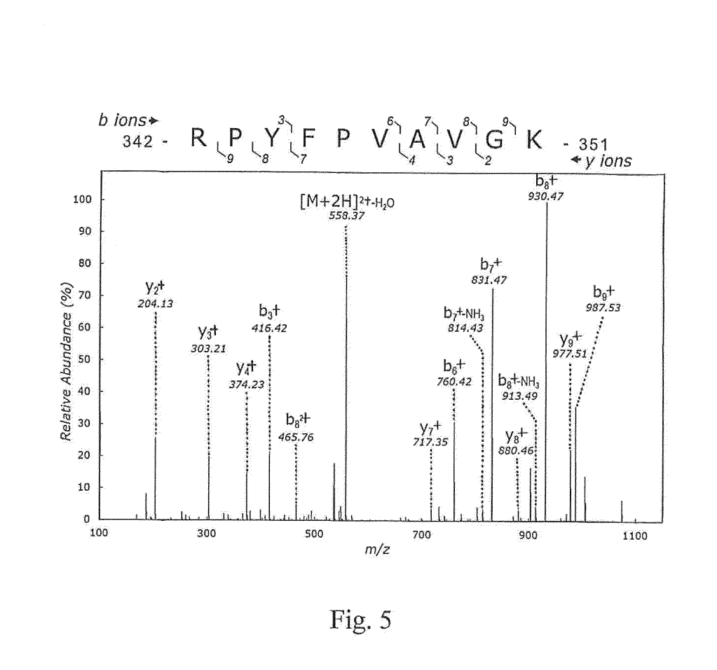

From the tryptic digest of 40 kDa species, LC-MS/MS analyses showed that almost all of the peptides resulted from tryptic cleavage. Only one peptide, i.e. ³³²HGGLYHENM³⁴⁰ (SEQ ID NO:4), had an unusual C-terminus (FIG. 3). LC-MS/MS analyses of its Arg-C digest showed another peptide with the unique terminal Met-340, i.e. ³²⁰CTLPKCDYPDIKHGGLYHENM³⁴⁰ (FIG. 4; SEQ ID NO:5). Altogether, these data indicate that Met-340 is the carboxyl end of 40 kDa CFH species (FIG. 6). On the other hand, LC-MS/MS analyses showed that a tryptic peptide with the primary structure ³⁴²RPYFPVAVGK³⁵¹ (SEQ ID NO:6) sat at the N-terminal end of the 140 kDa polypeptide. Since the same peptide was identified in the Lys-C digest (FIG. 5), these data indicate that Arg-342 is the amino end of the 140 kDa CFH polypeptide. Notably, Arg-341 was never uncovered in any digest of either 40 kDa or 140 kDa CFH species. Thus, we conclude that proteolytic removal of Arg-341 is the event leading to generation of the two CFH fragments in breast cancer patients (FIG. 6).

This notion is consistent with the fact that there exists a disulfide bond between the thiol groups of Cys-325 and Cys-374, which are located in 40 and 140 kDa species, respectively. This C325-C374 linkage is the molecular force keeping the two proteolytic fragments connected under non-reducing conditions, while its disruption by reducing agents could lead to the concurrent appearance of 40- and 140 kDa fragments (FIG. 2).

### Example 3

### Specific Proteolytic Fragments of Complement Factor H as Biomarkers of Breast Cancer

Considering that 40 kDa and 140 kDa species were produced through proteolytic removal of Arg-341, two new polypeptide ends should be generated within complement factor H. As biochemical analyses of these two ends might help distinguish breast cancers from other health conditions, these two end structures are putative novel breast cancer biomarkers. The C-terminal end (i.e. the C-terminal Met residue) of the small proteolytic fragment is designated as breast cancer proteolytic marker 1 (BCPM1) and the N-terminal end (i.e. the N-terminal Arg residue) of the large fragment is named as breast cancer proteolytic marker 2 (BCPM2).

To facilitate the validation of BCPM1 and BCPM2 as true breast cancer biomarkers, we first synthesized peptides corresponding to these two ends and used them for production of rabbit antisera. The polyclonal antibodies targeting each end (i.e. the C-terminal Met residue in the 40 kDa fragment and the N-terminal Arg residue in the 140 kDa fragment) were isolated using affinity purification methods.

Preparation of Rabbit Polyclonal Antibodies

Antibodies against BCPM1 were raised in rabbit using a synthetic peptide HGGLYHENM with a carboxylic acid group at the terminal Met. BCPM2 antibodies were generated by immunization of rabbits with a synthetic peptide RPYFPVAVGK with an amine group at the N-terminal Arg. Antibodies were then affinity-purified and tested for their specificity using dot blot methods as well as western blotting. Two specific antibodies, i.e. BCPM1 antibodies specifically targeting the C-terminal Met residue of the 40 kDa fragment and BCPM2 antibodies specifically targeting the N-terminal Arg residue in the 140 kDa fragment, were obtained and used in the subsequent western blot analyses.

Western Blot Analyses

BCMP1 and BCMP2 were used as primary antibodies for staining of plasma proteins from a normal individual and a breast cancer patient resolved with SDS-PAGE.

Results and Discussion

As mentioned, specific polyclonal anti-BCPM1 were generated to recognize the C-terminus of the 40 kDa proteolytic fragment while specific polyclonal anti-BCPM2 were for recognition of the N-terminus of the 140 kDa species. Using western blot analyses, we found that anti-BCPM1 could recognize one species, whose molecular mass is about 40 kDa, in the plasma from breast cancer patients. According to its molecular size, we conclude that this antibody can react with the BCPM1 structure. Since there were no other signals in these samples, it is concluded that anti-BCPM1 has very high specificity in recognition of its target epitope (i.e. the C-terminus of the 40 kDa proteolytic fragment). Likewise, we used anti-BCPM2 to analyze the same set of plasma samples with western blotting, which only revealed one single band of about 140 kDa. The molecular mass of this signal strongly suggests that we have successfully generated the antibodies against BCPM2. The high specificity of anti-BCPM2 was evidenced by the observation that very little or no signal was seen in other parts of the gel lane.

### Example 4

### Western Blot Analysis of Plasma Samples using BCPM1 and BCPM2 Antibodies

We further used the specific BCPM1 and BCPM2 antibodies to analyze the presence of the specific biomarker BCPM1 and BCPM2 in plasma samples from normal subjects or patients with breast cancer in different stages or other types of cancer. Table 1 shows the results.

TABLE 1
Results of western blot analyses of plasma samples from human
subjects with various health conditions using BCPM1 or BCPM 2
antibodies

| Sample (Stage) | Positive number | Total number | Positive % |
|---|---|---|---|
| Breast cancer (0) | 11 | 75 | 15 |
| Breast cancer (I) | 15 | 74 | 20 |
| Breast cancer (II) | 10 | 46 | 22 |
| Breast cancer (III) | 10 | 48 | 21 |
| Breast cancer (IV) | 13 | 48 | 27 |
| Normal-♀ | 0 | 20 | 0 |
| Normal-§ | 0 | 9 | 0 |
| Ovarian cancer | 0 | 4 | 0 |
| Colorectal cancer | 0 | 4 | 0 |
| Oral cancer | 0 | 6 | 0 |
| Gastric cancer | 0 | 6 | 0 |
| Diabetes Mellitus | 0 | 2 | 0 |
| | | Total: 342 | |

As shown in Table 1, the specific BCPM1 or BCPM2 antibodies, targeting the C-terminal Met residue of the 40 kDa fragment and the N-terminal Arg residue in the 140 kDa fragment respectively, successfully detected the breast cancer patients at various stage (from stage 0 to IV), but not normal individuals or patients with other types of cancer.

We demonstrated that the specific proteolytic cleavage of complement factor H at Arg-341, resulting two separate proteolytic fragments, i.e. 40 kDa and 140 kDa fragments, is a unique event specifically present in breast cancer patients, and thus can be used as a biomarker for screening for breast cancer. Therefore, the present invention for the first time provides a screening method for breast cancer, which can identify a patient as having, suspected of having, or at risk for breast cancer, at an early stage, i.e. before any symptom develops or a further physical exam or treatment for breast cancer is to be performed.

### Example 5

### ELISA Experiment for Breast Cancer Biomarker Detection

5.1 Material and Methods-Sandwich ELISA method

Capture antibody (either anti-BCPM1 or Anti-BCPM2) was coated in the bottom of the well of 96-well plate and the well was rinsed with PBST (PBS containing Tween 20). Serially diluted antigen (synthetic peptide) or sera from patients were applied to the well and reacted with coated capture antibody. The detection antibody conjugated with HRP (horseradish peroxidase) was then added to the well and reacted with antigen or sera from patients. HRP reacted with TMB (3′,3′,5′5′-tetramethylbenzidine) for 20 minutes and a stop reaction solution was added. The absorbance with 450 nm wavelength of sample solutions in each well (containing antigen or sera with different stages of breast cancer patients) was detected.

5.2 Results

The absorbance of 450 nm of sera (obtained from breast cancer patients with phase Ito phase IV), detected with sandwich ELISA kits, exhibited with higher value than those from sera obtained from normal people without breast cancer.

5.3 Conclusions

The innovation regarding two new biomarkers and the corresponding two new antibodies (both or either one anti-BCPM1 or anti-BCPM2), designed for breast cancer diagnostic kits, could be applied for screening of the occurrence of breast cancer.

### Sequence Information

Complement factor H (Homo sapiens)-Full length
(SEQ ID NO: 1)
MRLLAKIICL MLWAICVAED CNELPPRRNT EILTGSWSDQ TYPEGTQAIY KCRPGYRSLG 60
NVIMVCRKGE WVALNPLRKC QKRPCGHPGD TPFGTFTLTG GNVFEYGVKA VYTCNEGYQL 120
LGEINYRECD TDGWTNDIPI CEVVKCLPVT APENGKIVSS AMEPDREYHF GQAVRFVCNS 180
GYKIEGDEEM HCSDDGFWSK EKPKCVEISC KSPDVINGSP ISQKIIYKEN ERFQYKCNMG 240
YEYSERGDAV CTESGWRPLP SCEEKSCDNP YIPNGDYSPL RIKHRTGDEI TYQCRNGFYP 300
ATRGNTAKCT STGWIPAPRC TLKPCDYPDI KHGGLYHENM RRPYFPVAVG KYYSYYCDEH 360
FETPSGSYWD HIHCTQDGWS PAVPCLRKCY FPYLENGYNQ NYGRKFVQGK SIDVACHPGY 420
ALPKAQTTVT CMENGWSPTP RCIRVKTCSK SSIDIENGFI SESQYTYALK EKAKYQCKLG 480
YVTADGETSG SITCGKDGWS AQPTCIKSCD IPVFMNARTK NDFTWFKLND TLDYECHDGY 540
ESNTGSTTGS IVCGYNGWSD LPICYERECE LPKIDVHLVP DRKKDQYKVG EVLKFSCKPG 600
FTIVGPNSVQ CYHFGLSPDL PICKEQVQSC GPPPELLNGN VKEKTKEEYG HSEVVEYYCN 660
PRFLMKGPNK IQCVDGEWTT LPVCIVEEST CGDIPELEHG WAQLSSPPYY YGDSVEFNCS 720
ESFTMIGHRS ITCIHGVWTQ LPQCVAIDKL KKCKSSNLII LEEHLKNKKE FDHNSNIRYR 780
CRGKEGWIHT VCINGRWDPE VNCSMAQIQL CPPPPQIPNS HNMTTTLNYR DGEKVSVLCQ 840
ENYLIQEGEE ITCKDGRWQS IPLCVEKIPC SQPPQIEHGT INSSRSSQES YAHGTKLSYT 900
CEGGFRISEE NETTCYMGKW SSPPQCEGLP CKSPPEISHG VVAHMSDSYQ YGEEVTYKCF 960
EGFGIDGPAI AKCLGEKWSH PPSCIKTDCL SLPSFENAIP MGEKKDVYKA GEQVTYTCAT 1020
YYKMDGASNV TCINSRWTGR PTCRDTSCVN PPTVQNAYIV SRQMSKYPSG ERVRYQCRSP 1080
YEMFGDEEVM CLNGNWTEPP QCKDSTGKCG PPPPIDNGDI TSFPLSVYAP ASSVEYQCQN 1140
LYQLEGNKRI TCRNGQWSEP PKCLHPCVIS REIMENYNIA LRWTAKQKLY SRTGESVEFV 1200
CKRGYRLSSR SHTLRTTCWD GKLEYPTCAK R 1231
Complement factor H (Homo sapiens)-40 kDa fragment
(SEQ ID NO: 2)
MRLLAKIICL MLWAICVAED CNELPPRRNT EILTGSWSDQ TYPEGTQAIY KCRPGYRSLG 60
NVIMVCRKGE WVALNPLRKC QKRPCGHPGD TPFGTFTLTG GNVFEYGVKA VYTCNEGYQL 120
LGEINYRECD IDGWINDIPI CEVVKCLPVT APENGKIVSS AMEPDREYHF GQAVRFVCNS 180
GYKIEGDEEM HCSDDGFWSK EKPKCVEISC KSPDVINGSP ISQKIIYKEN ERFQYKCNMG 240
YEYSERGDAV CTESGWRPLP SCEEKSCDNP YIPNGDYSPL RIKHRTGDEI TYQCRNGFYP 300
ATRGNTAKCT STGWIPAPRC TLKPCDYPDI KHGGLYHENM 340
Complement factor H (Homo sapiens)-140 kDa fragment
(SEQ ID NO: 3)
RPYFPVAVGK YYSYYCDEHF ETPSGSYWDH IHCTQDGWSP AVPCLRKCYF PYLENGYNQN 60
YGRKFVQGKS IDVACHPGYA LPKAQTTVTC MENGWSPTPR CIRVKTCSKS SIDIENGFIS 120
ESQYTYALKE KAKYQCKLGY VTADGETSGS ITCGKDGWSA QPTCIKSCDI PVFMNARTKN 180
DFTWFKLNDT LDYECHDGYE SNIGSTIGSI VCGYNGWSDL PICYERECEL PKIDVHLVPD 240
RKKDQYKVGE VLKFSCKPGF TIVGPNSVQC YHFGLSPDLP ICKEQVQSCG PPPELLNGNV 300
KEKTKEEYGH SEVVEYYCNP RFLMKGPNKI QCVDGEWTTL PVCIVEESTC GDIPELEHGW 360
AQLSSPPYYY GDSVEFNCSE SFTMIGHRSI TCIHGVWTQL PQCVAIDKLK KCKSSNLIIL 420
EEHLKNKKEF DHNSNIRYRC RGKEGWIHTV CINGRWDPEV NCSMAQIQLC PPPPQIPNSH 480
NMTTTLNYRD GEKVSVLCQE NYLIQEGEEI TCKDGRWQSI PLCVEKIPCS QPPQIEHGTI 540
NSSRSSQESY AHGTKLSYTC EGGFRISEEN ETTCYMGKWS SPPQCEGLPC KSPPEISHGV 600
VAHMSDSYQY GEEVTYKCFE GFGIDGPAIA KCLGEKWSHP PSCIKTDCLS LPSFENAIPM 660
GEKKDVYKAG EQVTYTCATY YKMDGASNVT CINSRWTGRP TCRDTSCVNP PTVQNAYIVS 720
RQMSKYPSGE RVRYQCRSPY EMFGDEEVMC LNGNWTEPPQ CKDSTGKCGP PPPIDNGDIT 780
SFPLSVYAPA SSVEYQCQNL YQLEGNKRIT CRNGQWSEPP KCLHPCVISR EIMENYNIAL 840
RWTAKQKLYS RTGESVEFVC KRGYRLSSRS HTLRTTCWDG KLEYPTCAKR 890

What is claimed is:

1. A method, comprising:
(a) obtaining a biological sample from a subject; and
(b) detecting a cleaved form of a complement factor H(CFH) protein in the sample by a mass spectrometric assay or an immunoassay.

2. The method of claim 1, wherein the cleaved form of CFH is detected with an agent that specifically binds to the cleaved form of CFH.

3. The method of claim 2, wherein the agent specifically binds to the cleaved form of CFH is an antibody.

4. The method of claim 3, wherein the antibody specifically binds to (i) a peptide of SEQ ID NO:2, or (ii) a peptide of SEQ ID NO:3.

5. The method of claim 4, wherein the antibody binds to an epitope comprising the C-terminal Met residue of SEQ ID NO:2 or an epitope comprising the N-terminal Arg residue of SEQ ID NO:3.

6. The method of claim 3, wherein the antibody does not bind to the intact form of CFH.

7. The method of claim 1, wherein the biological sample is a body fluid sample, a tissue sample, or a biopsy sample.

8. The method of claim 7, wherein the biological sample is a body fluid sample selected from the group consisting of a blood sample, a plasma sample, a heparinized plasma sample, an EDTA-plasma sample, a serum sample, a urine sample, a saliva sample, a tear sample, a cerebrospinal fluid sample, and an ascites sample.

9. The method of claim 1, further comprising identifying the subject as having breast cancer or at risk for breast cancer, if the level of the cleaved form of CFH in the sample is elevated as compared to a reference level.

10. The method of claim 9, wherein presence of the cleaved form of CFH in the sample is indicative of occurrence or risk for breast cancer.

11. The method of claim 9, further comprising treating the subject with an anti-breast cancer therapy, if the subject is identified as having breast cancer, wherein the anti-breast cancer therapy is surgery, radiotherapy, or chemotherapy.

12. The method of claim 11, wherein the chemotherapy involves an anti-breast cancer drug selected from the group consisting of Abraxane, Anastrozole, Arimidex, Aromasin, Avastin, Docefrez, Docelaxel, Ellence, Epirubicin, Eribulin, Exemestane, Fareston, Faslodex, Femara, Fulvestrant, Gemcitabine, Gemzar, Halaven, Herceptin, Lxabepiline, Lxempra, Lapatinib, Letrozole, Megestrol, Paclitaxel, Tamoxifen, Taxotere, Toremifene, Trastuzumab, and Tykerb.

13. The method of claim 9, further comprising applying a further breast cancer diagnostic assay to the subject to confirm breast cancer occurrence.

14. A method for monitoring progression of breast cancer in a breast cancer patient, the method comprising:
(a) obtaining a first biological sample from the patient at a first time point,
(b) obtaining a second biological sample from the patient at a second time point, which is later than the first time point,
(c) measuring the levels of a cleaved form of a complement factor H(CFH) protein in the first and second biological samples by a mass spectrometric assay or an immunoassay; and
(d) determining breast cancer progression in the patient based on the levels of the cleaved form of CFH in the first and second biological samples, wherein an elevated level of the cleaved form of CFH in the second biological sample as compared to that in the first biological sample is indicative of breast cancer progression.

15. The method of claim 14, wherein the patient is subjected to an anti-breast cancer therapy and the first and second biological samples are obtained either before and after the treatment or during the course of the treatment.

16. The method of claim 15, further comprising assessing efficacy of the anti-breast cancer therapy on the patient, wherein a decrease of the level of the cleaved form of CFH after the treatment or over the course of the treatment indicates that the therapy is effective on the patient.

17. The method of claim 14, wherein the cleaved form of CFH is detected with an agent that specifically binds to the cleaved form of CFH.

18. The method of claim 17, wherein the agent specifically binds to the cleaved form of CFH is an antibody.

19. The method of claim 18, wherein the antibody specifically binds to (i) a peptide of SEQ ID NO:2, or (ii) a peptide of SEQ ID NO:3

20. The method of claim 19, wherein the antibody binds to an epitope comprising the C-terminal Met residue of SEQ ID NO:2 or an epitope comprising the N-terminal Arg residue of SEQ ID NO:3.

21. The method of claim 18, wherein the antibody does not bind to the intact form of CFH.

22. The method of claim 14, wherein the biological sample is a body fluid sample, a tissue sample, or a biopsy sample.

23. The method of claim 22, wherein the biological sample is a body fluid sample selected from the group consisting of a blood sample, a plasma sample, heparinized plasma sample, EDTA-plasma sample, a serum sample, a urine sample, a saliva sample, a tear sample, a cerebrospinal fluid sample, and an ascites sample.

24. A polypeptide or a complex thereof, wherein the polypeptide comprises an amino acid sequence at least 90% identical to that of a cleaved form of CFH.

25. The cleaved form of CFH of claim 24, wherein the cleaved form of CFH is SEQ ID NO:2, SEQ ID NO:3, or a complex form thereby.

26. A prepared monoclonal antibody specifically binding to a cleaved form of a complement factor H(CFH) protein.

27. The antibody of claim 26, which specifically binds to peptide fragment of SEQ ID NO: 2 or 3.

28. The antibody of claim 27, wherein the antibody specifically binds to the C-terminal portion of SEQ ID NO: 2 or the N-terminal portion of SEQ ID NO: 3.

29. The antibody of claim 28, wherein the antibody binds to an epitope comprising the C-terminal Met residue of SEQ ID NO:2 or an epitope comprising the N-terminal Arg residue of SEQ ID NO:3.

30. The antibody of claim 26, wherein the antibody does not bind to the intact form of CFH.

31. The antibody of claim 26, wherein the antibody is a chimeric antibody or a humanized antibody.

32. A kit for performing the method of claim 9, comprising an antibody that specifically binds to a cleaved form of CFH and instructions for performing the method.

33. A method for treating a subject having breast cancer, comprising administering to said subject an amount of an antibody that specifically binds to a cleaved form of CFH, wherein the antibody is linked with an anti-cancer agent.

34. A method for performing an in vivo diagnosis in a subject in need, comprising administering to subject suspected of having or at risk for breast cancer an amount of an antibody that specifically binds to a cleaved form of CFH, wherein the antibody is linked with a detectable label.


SEQUENCE LISTING (7 sequences)

SEQ ID NO: 1 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 340 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENM

SEQ ID NO: 3 - protein, 890 aa, Homo sapiens
RPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 4 - protein, 9 aa, Homo sapiens
HGGLYHENM

SEQ ID NO: 5 - protein, 21 aa, Homo sapiens
CTLPKCDYPDIKHGGLYHENM

SEQ ID NO: 6 - protein, 10 aa, Homo sapiens
RPYFPVAVGK

SEQ ID NO: 7 - protein, 90 aa, Homo sapiens
ATRGNTAKCTSTGWIPAPRCTLKPCDYPDIKHGGLYHENMRRPYFPVAVGKYYSYYCDEHFETPSGSYWDHIHCTQDGWSPAVPCLRKCY